(19) United States
(12) Patent Application Publication
(10) Pub. No.: US 2017/0215470 A1
(43) Pub. Date: Aug. 3, 2017

(54) USE OF A COMPOSITION CONTAINING A LONG-CHAIN POLYOL AS A BASE FOR E-LIQUIDS

(71) Applicant: LABORATOIRES CERES, Poitiers (FR)

(72) Inventors: Antoine PICCIRILLI, Poitiers (FR); Vincent BONNARME, Saint Aigny (FR)

(73) Assignee: LABORATOIRES CERES, Poitiers (FR)

(21) Appl. No.: 15/323,854

(22) PCT Filed: Jul. 10, 2015
(86) PCT No.: PCT/FR2015/051919

§ 371 Date: Apr. 13, 2017

(30) Foreign Application Priority Data
Jul. 10, 2014 (FR) 14 56655

(51) Int. Cl. A24B 15/16; A61K 47/10; A61K 31/465; A24F 47/00

(52) U.S. Cl. CPC A24B 15/167; A24F 47/008; A61K 47/10; A61K 31/465

(57) ABSTRACT

Provided is a composition containing a polyol that has 4 to 8 carbon atoms and 2 hydroxyl functions that can be used as a liquid for electronic cigarettes. The disclosure further relates to a liquid composition for electronic cigarettes, the composition containing a polyol that has 4 to 8 carbon atoms and 2 hydroxyl functions, as well as at least one compound selected from among nicotine, a nicotine substitute and an aroma. The disclosure also relates to an electronic cigarette containing the composition.

### TECHNICAL AREA

The present invention relates to the use of a composition comprising a long-chain polyol as a liquid for an electronic cigarette. It also has as subject matter a composition of liquid for an electronic cigarette comprising a polyol as well as nicotine and/or at least one aroma as well as an electronic cigarette with a related device comprising this composition.

### BACKGROUND OF THE INVENTION

The market for the electronic cigarette is currently undergoing a significant development due to the fact that it allows the user to retain the ritual associated with the use of the cigarette without experiencing the deleterious effects of the harmful substances which it comprises.

The electronic cigarette or e-cigarette functions by electricity without combustion. It produces a mist of fine particles commonly called vapor or artificial smoke, visually resembling the smoke produced by the combustion of tobacco. This vapor can be aromatized (aroma of tobacco, mint, fruits, chocolate, etc.) and contains or does not contain nicotine. In e-cigarettes which are correctly manufactured and used, the aerosol contains, according to the available data, much fewer substances which are deleterious to health than tobacco smoke, in particular no solid particles no tar, no other carcinogenic substances and no carbon monoxide (CO).

The electronic cigarette has three main parts contained in a plastic or metallic envelope:

a battery,

a cartridge or reservoir containing a liquid called “e-liquid”, and

an atomizer.

The battery constitutes most of the time the largest part of the e-cigarette in the disposable products. In the reusable cigarette they are “low voltage” batteries (accumulators) which can be recharged by USB cable or by a charger. In the reusable e-cigarette the tube protecting the battery is screwed onto the cartridge containing the liquid. In certain models a luminous signal-usually a red or blue diode—is placed at the other end of the battery tube.

The storage device of the e-liquid can have the shape of a cartridge (generally of silicone, PMMA or stainless metal) or of a reservoir (in particular of PMMA/polyethylene, borosilicate glass or stainless metal) optionally completed with a device for collecting liquid by capillarity (in particular of silica, glass fibre, ceramic metallic tissue, nylon threads or borosilicate fibres) in contact with the vaporization system. The atomizer allows the conversion of the e-liquid into a mist simulating smoke. It is constituted by a metallic spiral or trellis which forms a heating resistance. It is more and more frequently integrated in the rechargeable cartridge. A microvalve sensitive to the partial vacuum caused by the inhaling or a contactor with manual release allows the supplying of the atomizer by the battery. The e-cigarette can be used once or reused.

The e-liquids used are primarily composed of the following constituents:

synthetic propylene glycol (approximately 65%)

glycerol (approximately 25%)

water (5 to 10%)

aromas and colorants (2 to 5%)

nicotine (0 to 20 mg/ml)

Certain e-liquids can also contain ethanol in a significant quantity (>1%).

Certain products can lack synthetic propylene glycol. The objective in this case is to be able to claim products with an exclusively vegetable origin. However, this objective is achieved at the cost of the longevity of the heating resistances, which very rapidly become dirty. Furthermore, the quality of the smoke emitted is far from being suitable as concerns the density of the vapor, and the organoleptic properties of the liquids are greatly modified because the freeing of the aromas in the absence of propylene glycol is rendered less immediate and is greatly modified at the organoleptic level. Furthermore, the exclusive use of glycerol requires the product to be charged with water in order to reduce the viscosity of the e-liquid and to the facilitate in this manner the filling of the e-cigarette. However, here too, the impact of a large content of water radically modifies the quality of the vapor emitted and leads to an excessive corrosion of the materials as well as to a rapid and excessive consumption of the e-liquid (more rapid vaporization). Furthermore, the presence of water requires taking drastic precautions at the industrial level in order to avoid any microbiological pollution (ex. sterile filtration, addition of a preservative agent). Finally, another problem associated with the exclusive use of glycerol is the fact that this compound is clearly less vaporizable than propylene glycol, in such a manner that its vaporization necessitates a heating temperature which is distinctly greater and capable of bringing about its degradation and the formation of undesirable by-products such as acrolein.

Consequently, the use of synthetic propylene glycol in a quantity greater than glycerol is most frequently preferred, which does not allow the producers to claim a natural origin for their products. Furthermore, the propylene glycol is obtained according to a process which is included among the most energy-consuming ones in petrochemistry and therefore has a great environmental impact (Eissen & coll., Chem. Int. Ed. 2002, 41, 414-436) which is expressed by a great consumption of energy and a significant production of volatile organic compounds (COVs) and waste. Furthermore, synthetic propylene glycol is obtained from propylene oxide according to a continuous hydration process according to the following scheme:

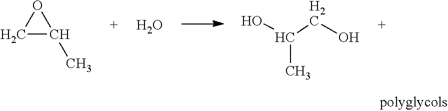

The production of propylene glycol is accompanied by the formation of secondary products (de-, and tri- and tetrapropylene glycols) and of non-converted propylene oxide Petrochemical Processes: Major Oxygenated, Chlorinated and Nitrated Derivatives—Alain Chauvel, Gille Lefebvre—Editions TECHNIP—p. 26), as illustrated below:

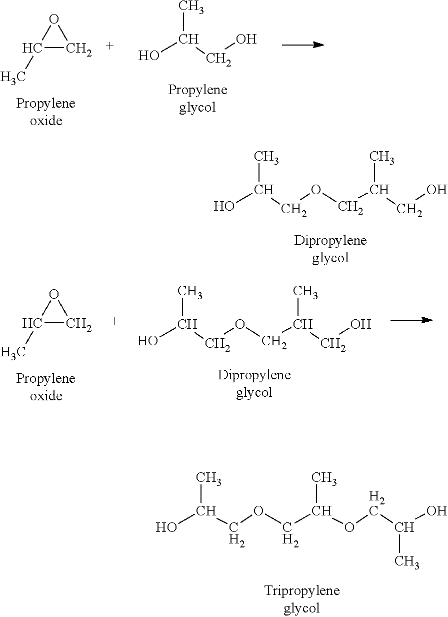

Consequently, after purification the lesser and recurrent organic impurities of propylene glycol are di- and tripropylene glycol, as well as propylene oxide, whose residual content is according to the producers on the order of 5 to 10 ppm (Propylene Glycol—CIR Expert Panel, Jun. 28-29 2010—Draft Report). Now, propylene oxide is classified by the North-America and European environmental agencies as a carcinogenic compound and mutagenic in animals and probably carcinogenic in humans. Consequently, it is advisable to strictly limit the exposure to this compound. Also, the Report and Notice of Experts about the E-Cigarette published by the French Office of Prevention of Nicotine Poisoning (OFT) in May 2013 insists on the necessity of guaranteeing the absence of carcinogenic contaminants in e-liquids. In fact, it is advisable to avoid the presence of a toxic compound such as propylene oxide and to a lesser extent the presence of organic impurities belonging to the family of glycol ethers, which is greatly discredited toxicologically and which alters the quality of e-liquids in the manner of di-and tripropylene glycols.

A solution to the previously cited problems was proposed in the application WO 2013/088230. It consists in substituting synthetic propylene glycol with propylene glycol of vegetable origin obtained by the catalytic hydrogenation of sorbitol coming from corn. Propylene glycol is associated with glycol of vegetable origin, with nicotine which can be extracted from tobacco leaves and possibly with aromas of a natural origin in order to obtain an e-liquid of entirely vegetable origin.

If this solution effectively allows overcoming the disadvantages associated with the use of synthetic propylene glycol, it has been demonstrated that the density of the vapor and the aromatic potency produced by these e-liquids of vegetable origin can be improved by replacing the propylene glycol by a long-chain polyol and that this effect was particularly remarkable in the absence of glycerol or in a composition of e-liquid with a low content of glycerol. While allowing the glycerol to be eliminated, the using of a long-chain polyol furthermore contributes to protecting the heating device of the electronic cigarettes by eliminating the phenomenon of rapid fouling observed in the presence of glycerol. Another advantage associated with the absence of glycerol is that the vapor produced is cleansed of toxic and carcinogenic impurities stemming from the thermal decomposition of the glycerol.

Another advantage of using a long-chain polyol is that it allows the formulation of nicotine in the absence of water. In fact, it is known that the nicotine base in solution in water is transformed into protonated nicotine. Now, the protonated form is distinctly less bio-assimilatable than base nicotine. Consequently, the use of a long-chain polyol in the absence of water allows e-liquids to be designed which have a better performance as regards the nicotinic withdrawal and are safer because they are less concentrated with nicotine, an alkaloid whose acute toxicity is very elevated.

Furthermore, it was observed that a long-chain polyol allowed the obtention of e-liquids without nicotine, which creates the tingling sensation in the throat (or “throat hit”) typically felt by the user of a classic cigarette during the passage of nicotine into the mouth. Up to the present, this effect, which was very desired by the users of electronic cigarettes, was only obtained by adding a few drops of a product based on propylene glycol, glycerol and aromas (E-Liquide Flash® of FLAVOUR ART). However, the latter has all the above-cited disadvantages associated with the use of propylene glycol and glycerol. The long-chain polyols are basically of a synthetic origin but some are of a renewable origin like 1,4 butane diol, 2,3 butane diol, 1,2 pentane diol.

### SUMMARY OF THE INVENTION

The present invention therefore has as subject matter the use of a composition containing a polyol comprising 4 to 8 carbon atoms and 2 hydroxyl functions as liquid of an electronic cigarette.

It also has as subject matter a composition of liquid for an electronic cigarette containing a polyol comprising 4 to 8 carbon atoms and 2 hydroxyl functions as well as at least one compound selected from nicotine, a nicotine substitute and an aroma.

It also has as subject matter an electronic cigarette comprising this composition.

It has as subject matter the use of a long-chain polyol in an electronic cigarette liquid comprising or not comprising nicotine in order to improve the tingling of the throat felt by a user of this liquid and/or the ease of inhaling the vapor produced by this liquid.

It also has as subject matter the use of a long-chain polyol in an electronic cigarette liquid in order to reinforce the aromatic potency or to limit or eliminate the formation of coproducts of thermolysis or in order to increase the density of the vapor.

Finally, it has as subject matter the use of a polyol comprising 4 to 8 carbon atoms and 2 hydroxyl functions in an electronic cigarette liquid comprising nicotine in order to improve the bioavailability of the nicotine.

### DETAILLED DESCRIPTION OF EMBODIMENTS

In the present application “electronic cigarette” designates the unit of the devices provided with electrical means producing vapor and delivering nicotine and/or an aroma. This definition therefore comprises in particular the personal vaporizers (VP), the electronic systems for the distribution of nicotine (or ENDS for “Electronic Nicotine Delivery System”, or ENDD for “Electronic Nicotine Delivery Device”), as well as electronic cigars, electronic pipes and electronic chichas, cigarettes based on heated tobacco or containing a tobacco aroma obtained by maceration.

Composition “of vegetable origin” denotes a composition comprising at least 95% of bio-sourced carbon such as determined by the ASTM standard D6866-12 (Standard Test Methods for Determining the Biobased Content of Solid, Liquid, and Gaseous Samples Using Radiocarbon Analysis).

A previously indicated, the invention relates to the use of a composition comprising a polyol containing 4 to 8 carbon atoms and 2 hydroxyl functions as liquid for an electronic cigarette (“e-liquid” hereinafter).

Moreover, as previously indicated, the invention also relates to:

The use of a long-chain polyol in an electronic cigarette liquid comprising or not comprising nicotine in order to improve the tingling of the throat felt by a user of this liquid and/or the ease of inhaling the vapor produced by this liquid, and

the use of a long-chain polyol in an electronic cigarette liquid for reinforcing the aromatic potency or for limiting or eliminating the formation of coproducts of thermolysis or for increasing the density of the vapor.

In the present invention the term “polyol comprising 4 to 8 carbon atoms and 2 hydroxyl functions” denotes a compound comprising 4 to 8 carbon atoms, preferably 4, 5, 6, 7 or 8 carbon atoms and 2 hydroxyl functions (—OH).

In the present invention the term long-chain polyol denotes a compound with at least one of the following characteristics:

A hydrocarbonated compound comprising 2, 3 or 4 hydroxyl functions, preferably 2;

A compound comprising 4 to 12 carbon atoms, preferably 4, 5, 6, 7, 8, 9 or 10 carbon atoms;

A linear or cyclic compound, preferably linear;

A compound of vegetable or synthetic origin, preferably vegetable.

The polyol comprising 4 to 8 carbon atoms and 2 hydroxyl functions or the long-chain polyol can be synthetic or, according to a preferred embodiment of the invention, it can be obtained from vegetable raw materials and designated here by “polyol of vegetable origin”.

The polyol with C4-C8 comprising 2 hydroxyl groups according to the invention belongs in a non-limiting manner to the group constituted by 1,2-butane diol, 1,3-butane diol, 1,4-butane diol, 2-methyl-1, 3-propane diol, 2-methyl-1, 3-butane diol, 2-methyl-1, 4-butane diol, 2 ethyl-1,3-propane diol, 1,2-pentane diol, 1,3-pentane diol, 1-4-pentane diol, 1-5-pentane diol, 1-2-hexane diol, 1-3-hexane diol, 1-4-hexane diol, 1-5-hexane diol, 1-6-hexane diol, 2-methyl-1, 3-pentane diol, 2-methyl-1, 4-pentane diol, 2-methyl-1, 5-pentane diol, 2-ethyl-1, 3-butane diol, 2-ethyl-1, 4-butane diol, 1,2-octane diol, 1,3-octane diol, 1,4-octane diol, 1,5-octane diol, 1,6-octane diol, 1,7-octane diol, 1,8-octane diol, 2-methyl-1, 4-heptane diol, 2-methyl-1, 5-heptane diol, 2-methyl-1,6-heptane diol, 2-methyl-1, 7-heptane diol, 3-methyl-1, 2-heptane diol, 3-methyl-1, 3-heptane diol, 3-methyl-1, 4-heptane diol, 3-methyl-1, 5-heptane diol, 3-methyl-1, 6-heptane diol, 3-methyl-1, 7-heptane diol, 2-ethyl-1, 3-hexane diol, 2-ethyl-1, 4-hexane diol, 2-ethyl-1, 5-hexane diol, 2-ethyl-1, 6-hexane diol, 3-ethyl-1, 2-hexane diol, 3-ethyl-1, 3-hexane diol, 3-ethyl-1, 4-hexane diol, 3-ethyl-1,5-hexane diol, 3-ethyl-1, 6-hexane diol, 1,4-cyclohexane diol and methyl-2-1,4-cyclohexane diol.

The polyol is preferably 1,2-butane diol, 1,3-butane diol, 1,4-butane diol, 1,2-pentane diol, 1,2-hexane diol, 1,2-heptane diol or 1,2-octane diol.

The composition used according to the invention can furthermore comprise propylene glycol. The latter can be of synthetic or vegetable origin (that is, obtained from vegetable raw materials). In the latter case, which is preferred, the propylene glycol can be obtained in particular by the hydrogenolysis of sorbitol or of vegetable glycerol (New and Future Developments in Catalysis: Catalytic Biomass Chemistry—S. Suib Editor/Elsevier—2013, pp. 13-17) or by hydrogenation of vegetable lactic acid (J. Van Haver en & coll., Bulk Chemicals from Biomass. BioFPR, Nov. 1, 2007. Pp. 41-57).

The biosourced glycerol used to produce the propylene glycol can be of animal or vegetable origin, preferably vegetable. The vegetable glycerol comes from the hydrolysis (acid or basic) of vegetable oils or from their alcoholysis (transesterification). These oils belong in a non-limiting manner to the groups soy oil, palm oil, palm kernel oil, copra oil, rape oil, sunflower oil, corn germ oil, cotton oil, olive oil, sesame oil, rice bran oil, flax oil, castor oil, avocado oil, peanut oil, safflower oil, raisin seed oil, pine oil (tall oil). Glycerol from vegetable varieties which are not genetically modified are preferred such as palm oil, rape oil, sunflower oil or copra oil.

The sorbitol or the biosourced lactic acid used to produce the propylene glycol of vegetable origin generally comes from sugary or starchy vegetables such as sugar cane, corn, wheat, potato, sugar beet, rice or sorghum. Sorbitol or lactic acid from non-genetically modified vegetable varieties is preferably used such as sugar cane or the beet. Even better, the sorbitol or the lactic acid is obtained from non-food, lignocellulosic biomasses such as wood, straw, palm bunches, bagasse and non-genetically modified corn cobs.

Note that the previously cited processes allow obtaining not only propylene glycol but also 1,3-propane diol (PDO) as coproduct whose proportions can be adjusted by appropriately selecting the reaction conditions (Niir Dyana bt Saar—Dissertation submitted in partial fulfillment of the requirements for the Bachelor of Engineering (Hons) Chemical Engineering, PETRONAS University of Technology, May, 2013).

The propylene glycol can be from 2 to 50% by weight, preferably 10 to 40% by weight, more preferably 20 to 30% by weight relative to the total weight of the composition.

It is preferable according to the invention that the composition used as e-liquid does not contain or contains only a little glycerol, that is, it contains 0 to 40% by weight of glycerol, preferably 0 to 20% by weight of glycerol, for example, 0 to 5% by weight glycerol or 5 to 20% of glycerol relative to the total weight of the composition. In fact, it was observed as indicated above, that the absence of glycerol allowed the avoidance of the formation of undesirable byproducts during the heating of the glycerol. It was precisely determined that at the temperature achieved by the resistance of an electronic cigarette the glycerol decomposed to acrolein (Cordoba & coll. Proceedings of COBEM 2011—21^{st} Brazilian Congress of Mechanical Engineering, 24-28 October, 2011, Natal, R N, Brazil Metzger Brian; Glycerol Combustion—Thesis of the University of North Carolina, 1 Aug. 2007) a highly toxic compound in a very low concentration (Goniewicz & coll. Levels of Selected Carcinogens and Toxicants in Vapour from Electronic Cigarettes—TC Online First, published 6 Mar. 2013 under 10.1136/tobaccocontrol-2012-050859). It was also noted in a surprising manner that the absence of glycerol allowed the vapor density and the aromatic potency of the electronic cigarette to be distinctly increased. When it is present, the glycerol advantageously is of vegetable origin and is obtained according to the previously described processes.

The term aromatic potency of a product denotes the olfactory and taste perception of the product, that is, its flavor, a term which includes the savor, the astringency, the pseudo-heat and the aroma of the product. The aromatic potency is evaluated according to olfactory and taste protocols by a panel of testers.

The composition can also contain synthetic 1,3-propane diol or, according to a preferred embodiment of the invention it can be obtained from raw vegetable materials and designated here by “1,3-biopropane diol of vegetable origin”. The 1,3-biopropane diol of vegetable origin can be obtained by the fermentation of glucose in the presence of a native or genetically modified bacterium selected in particular from the strains of Klebsiella (in particular pneumoniae), Clostridium (in particular butyricum) Citrobacter (in particular freundii), Serratia and Escherichia coli, preferably Escherichia coli, and more preferably Escherichia coli K-12. An example of a genetically modified strain is described in the US 2012/258521 application. The biosourced glucose used for producing 1,3-propane diol generally comes from sugary or starchy vegetables such as sugar cane, corn, wheat, potato, sugar beet, rice or sorghum. The glucose preferably corns from non-genetically modified vegetable varieties such as sugar case or beets. Better yet, the glucose comes from non-food, lignocellulose biomasses such as wood, straw, palm bunches, bagasse and non-genetically modified corn cobs. The fermentation product can be recovered and the 1,3-biopropane diol purified by membrane filtration, electrodialysis, concentration or rectification, for example, or by a combination of these techniques. The 1,3-biopropane diol can in particular be purified by distillation, an operation which allows a purity of 99.8% to be achieved. The impurities present at the level of 0.2% are water and propanol-1 (Chatterjee & coll. Glycerol to Propylene Glycol/Department of Chemical & the Biomolecular Engineering Senior Design Reports (CBE), University of Pennsylvania—Apr. 12, 2011), a compound stripped of toxicity. 1,3-propane diol can represent 2 to 50% by weight, preferably 10 to 40% by weight, more preferably 20 to 30% by weight relative to the total weight of the composition.

Aside from the previously cited constituents, the composition used according to the invention also comprises at least one compound selected from nicotine, a substitute of nicotine (typically a non-addictive molecule but with a sensory effect close to that of nicotine) and an aroma.

The nicotine can be of a synthetic or vegetable origin and should preferably meet the valid purity criteria described in the American (USP) and European (PE) pharmacopoeias. It can in particular be extracted from tobacco leaves or obtained by chemical synthesis. The concentration of nicotine in the composition of the invention can range from 0 to 100 mg/ml, preferably from 2 to 25 mg/ml.

The aromas can also be aromas of a vegetable or synthetic origin such as the homologues in the food and/or pharmaceutical areas, in particular those listed in the UE regulation No. 872/2012dated 1 Oct. 2012 and in the valid American (USP) and European (PE) pharmacopoeias. The concentration of aromas can range from 0 to 30% by weight, preferably from 1 to 8% by weight, more preferably from 2 to 5% by weight relative to the total weight of the composition.

The composition used according to the invention can also comprise water and/or an alcohol such as ethanol and/or at least one colorant. The water and alcohol can each represent 0 to 20% by weight, preferably 1 to 10% by weight relative to the total weight of the composition. The colorants can be colorants of vegetable or synthetic origin such as the homologues in the food and or pharmaceutical areas in the particular those listed in the UE regulation No. 1331/2008 and in the valid American (USP) and European (PE) pharmacopoeias. The concentration of aromas can range from 0 to 30% by weight, preferably from 1 to 8% by weight, more preferably from 2to 5% by weight relative to the total weight of the composition.

However, it is preferred according to the invention that the composition does not comprise water with the exception of what might be contained in the raw materials which the composition comprises. In fact, the water can favor the development of pathogenic microorganisms of a microbial origin and its use generally necessitates the using of preservatives or of a sterilizing microfiltration. Furthermore, the addition of water to the e-liquids induces a transformation of the base nicotine to protonated nicotine. Now, the person skilled in the art knows that the protonated form of nicotine is distinctly less bioassimilable, and in fact less addictive than the base nicotine. Therefore, certain long-chain polyols allow the formulation of very liquid e-liquids without having to add water and in which the nicotine is present in the base form and in its highly bioavailable form, which distinctly improves the controlling of the delivering of the nicotine, in particular during nicotinic withdrawal.

The invention also relates to an electronic cigarette comprising the composition as described above. The latter is generally arranged in a cartridge integral with a receptacle protecting an electrical supply system connected to a device for atomizing the composition.

It also relates to the use of a long-chain polyol in an electronic cigarette liquid comprising or not comprising nicotine in order to improve the tingling in the throat felt by a user of this electronic cigarette and/or to make it easier to inhale the vapor produced by this liquid.

The invention also relates to the use of a long-chain polyol in an electronic cigarette liquid for reinforcing the aromatic potency.

It also relates to the use of a long-chain polyol in an electronic cigarette liquid in order to limit or eliminate the formation of thermolysis byproducts.

Finally, it relates to the use of a long-chain polyol in an electronic cigarette liquid for increasing the vapor density.

In the uses described above the long-chain polyol can be a compound comprising 4 to 8 carbon atoms, preferably 4, 5, 6, 7 or 8 carbon atoms and 2 hydroxyl functions.

According to an embodiment the long-chain polyol is 1,2-butane diol, 1,3-butane diol, 1,4-butane diol, 1,2-pentane diol, 1,2-hexane diol, 1,2-heptane diol or 1,2-octane diol.

Furthermore, it relates to the use of a polyol comprising 4 to 8 carbon atoms and 2 hydroxyl functions in an electronic cigarette liquid comprising nicotine for improving the bioavailability of the nicotine.

The invention will be better understood in view of the following examples which are only given only by way of illustration and do not have the goal of limiting the scope of the invention defined by the attached claims.

### EXAMPLES

### Example 1

Preparation and Analysis of a Composition Based on 1,3-Butane Diol and on Glycerol

Precisely 10.00 kg of 1,3-butane diol such as sold by the Kyowa company under the name 1,3-butylene glycol, cosmetic grade), 1.00 kilograms of vegetable glycerol (sold by the Oleon company under the reference Glycérine 4810, grade USP and PE pharmacopoeias), 450.0 g apple fruit aroma (sold by the Safisis company under the reference PT 128) and 114.50 g of vegetable nicotine (sold by the Alchem International company under the reference Nicotine Free Base, pharmaceutical grade nicotine>99%) are precisely mixed in a glass mixture provided with a mechanical agitation. The agitation of the mixture (50 revolutions/minute) is maintained for 20 minutes. A sampling of 500 g is made for analysis. Then, a measuring of the kinematic viscosity is carried out at 25° C. with the aid of a Houillon viscosimeter

tube m 943 with a coefficient of 0.35051.

Results

The viscosity at 20° C. is equal to 65.2 mm²/s.

### Example 2

Preparation and Analysis of a Composition Without Glycerol

The identical procedure as in example 1 is followed but with replacing the glycerol of vegetable origin by 1 kg of 1,3-butane diol (supplied by Kyowa) according to example 1. A measuring of the viscosity is then carried out under the same conditions as in example 1.

Results

The viscosity at 20° C. is equal to 101.2 mm²/s.

### Example 3 (Comparative)

Evaluation of the Efficiency of Different E-liquids

The identical procedure as in example 1 is followed but with replacing the 1,3-butane diol with synthetic propylene glycol supplied by the Dow company with the reference Dow® Propylene glycol, grade USP. A measuring of the viscosity is then carried out at 20° C.

Results

The viscosity at 20° C. is equal to 59.2 mm²/s.

In conclusion, it can be established that e-liquids based on 1,3-butane diol and associated or not associated with glycerol (examples 1 and 2) have viscosities quite comparable with that of a conventional e-liquid (example 3).

### Example 4

Evaluation of the Efficiency of Different E-liquids

The compositions of e-liquids prepared in the examples 1 to 3 are evaluated by a trained panel of 10 persons provided with a cigarette of the Joytech® brand and of the eCab™ model (model December 2013). Each reservoir is filled with an identical quantity of e-liquid (1 ml). Also, each panelist is blindfolded and performs a trial on the basis of 8 successive puffs spaced at 20 seconds and each induced by a heating of 2 seconds. The evaluation is based on the notation, on a scale of 1 to 10, of the criteria of vapor density and of the aromatic potency experienced. The passage from one product to another one is carried out by each panelist in the following manner: 5 minutes after the last inhalation the panelist rinses his mouth with the aid of 2 glasses of water of 100 ml and then quenches his thirst with 50 ml of water. A rest period between each 20 evaluation is fixed at 10 minutes.

The mean results obtained are collected in the following table:

| | Vapor density | Aromatic potency |
|---|---|---|
| Product | (grade 1 to 10) | (grade 1 to 10) |
| Example 1 | 7.2 ± 1.3 | 6.7 ± 1.5 |
| Example 2 | 7.9 ± 1.2 | 7.8 ± 1.2 |
| Example 3 | 6.8 ± 1.1 | 5.4 ± 1.1 |

It is clearly apparent that the 1,3-butane diol associated with the glycerol of vegetable origin (example 1) or not associated with the glycerol (example 2) allows the production of an e-liquid with a vapor density that is quite comparable, even superior to that of a conventional e-liquid (example 3). The e-liquids based on 1,3-butane diol allow the obtention of an aromatic potency that is superior to the one obtained with a conventional e-liquid (example 3).

### Example 4

Influence of the Nature of the Solvent on the Concentration of Base Nicotine in the Formulations of E-Liquids

Precisely 5.50 kg of propylene glycol of vegetable origin (rape) sold by the Oleon company under the reference Radianol® 4710, USP pharmaceutical grade), 4.00 kg vegetable glycerol (sold by the Oleon company under the reference Glycerine 4810, USP and PE pharmaceutical grade) 50 g of osmotic water and 162.6 g of vegetable nicotine (sold by the Alchem International company under the reference Nicotine Free Base, pharmaceutical grade nicotine>99%) are mixed in a glass mixer provided with a mechanical agitation. The agitation of the mixture (50 revolutions/minute) is maintained for 20 minutes. Product A is then obtained. A sampling of 200 g is made for analysis. The identical procedure for product A is followed for preparing the product B while replacing the propylene glycol and the vegetable glycerol by 9,837.4 g of 1,3-butane diol supplied by the Kyowa company. A sampling of 200 g of B is carried out for analysis.

An MNR spectrum of the proton (Magnetic Nuclear Resonance) is made on an apparatus of the Avance Brucker brand (500 MHz), of the products A, B, C, D dissolved in advance in D₂O (deuterated water). The objective is to measure the percentage of protonated nicotine in the products. This quantification by RMN of the proton is made on the basis of a calibration curve covering the concentration range of the protonated nicotine comprised at 5 and 95%.

The results are collected him in the following table:

| Product/measure | A | B |
|---|---|---|
| Concentration of protonated nicotine, % | 34 | 81 |

It is clearly apparent that by permitting the formulation of the e-liquids without water, 1,3-butane diol ensures the delivering of the nicotine in the base form (non-protonated) and therefore in a very bioavailable form.

### Example 5

Influence of the Nature of the Solvent on the Sensory Properties of the E-liquid and on the Ease of Inhaling the E-liquid

The compositions of e-liquids, products A and B and C (the latter corresponds to 1,3-butane diol without added nicotine), prepared according to example 4, are evaluated by a trained panel of 40 persons (sex masculine, age comprised between 25 and 49 years old), provided with a cigarette of the Joytech(tm) brand and with the eCab(tm) model (model of December, 2013). Each reservoir is filled with an identical quantity of e-liquid (1 ml).

Also, each panelist is blindfolded and performs a trial on the basis of 8 successive puffs spaced at 20 seconds and each induced by a heating of 2 seconds. The passage from a product to another one is made by each panelist in the following manner: 5 minutes after the last inhalation the panelist rinses his mouth with the aid of 2 glasses of water of 100 ml and then quenches his thirst with 50 ml of water. A rest period between each evaluation is fixed at 10 minutes. The evaluation is based on the notation, on a scale of 1 to 10, of the following criteria:

1) The sensation of the “throat hit”, that is, the effect of the internal tingling of the throat classically obtained when a smoker inhales a cigarette puff, which is also felt when a user of an electronic cigarette inhales him an e-liquid vapor rich in nicotine,

2) The ease of inhaling the vapor of the e-liquid.

The mean results obtained are collected in the following table:

| | Sensation of the “throat hit” | Ease of inhalation |
|---|---|---|
| Product | (grade 1 to 10) | (grade 1 to 10) |
| Product A | 6.4 ± 1.2 | 6.9 ± 1.4 |
| Product B | 8.9 ± 0.9 | 6.7 ± 1.1 |
| Product C | 8.1 ± 0.8 | 6.3 ± 1.2 |

It is clearly apparent that 1,3-butane diol associated with the nicotine (product B) induces an “hit throat” quite superior to a classic product constituted by glycerol, propylene glycol, water and nicotine (product A). Finally, it is very interesting to emphasize that 1,3-butane diol alone (product C) induces a very significant “hit throat” in the total absence of nicotine and significantly greater than the product A corresponding to a conventional e-liquid.

As regards the ease of inhaling vapor, the e-liquids A, B and C exhibit basically equivalent performances.

### Example 6

Evaluation of the Thermal Stability of the Formulations Based on 1,3-Butane Diol and Comparison to the Current Formulations Based on Propylene Glycol and Glycerol

The thermal stability of the 1,3-butane diol and of its mixture with nicotine was evaluated and compared to actual compositions based on propylene glycol (PG) and on vegetable glycerol (VG). The stability of the products and the possible interaction between the nicotine and 1,3-butane diol were evaluated by thermogravimetric analyses (TGA) and thermal differential analyses (TDA) on a Q600 TA Instrument apparatus. The thermogravimetric analysis is a method of thermal analysis in which the changes of the physical and chemical properties of the materials are measured as a function of the temperature. The TGA can supply information about the physical phenomena such as the vaporization or the combustion. Also, it is possible to obtain information about the chemical phenomena such as the dehydration or the decomposition. In the thermal differential analysis (TDA) the study material and an inert reference undergo identical thermic cycles. During the analysis the temperature difference between the sample and the reference is recorded. These temperature differences followed as a function of the rime relative to the inert reference inform us about the thermal phenomena undergone by the sample, whether exothermic or endothermic.

The TDA shows, by integration of the course of the heat flow, the values of enthalpies which are called endothermic when there is an absorption of energy, or exothermic when there is a release of energy in the form of heat. The endothermic character is associated with a change of state of the compound (ex. vaporization) whereas the exothermic character is induced by reactions of decomposition or by intra- and intermolecular chemical reactions. The thermogravimetric analyzer Q600 of TA INSTRUMENT was used to conjointly determine the vaporization temperatures of the products, the enthalpy values and the rate of products not vaporized at 350° C. The analysis conditions are as follows:

test size: 50 mg

open crucible under flow of air: 100 ml/min

rate of heating: 10° C./min

temperature range: 25 to 350° C. (temperature conditions representative of a normal use as well as of misuse of an electronic cigarette). The apparatus is cleaned between each analysis by being heated to 600° C. under a flow of air in order to eliminate any trace of non-vaporized residue deposited on the crucible, the balance arms or the kiln walls.

The results obtained are presented in the following table:

| | TDA | TDA | TGA |
|---|---|---|---|
| | vaporization | transformation | non-evaporated |
| | temperature | temperature | residue at |
| Product | (° C.) (1) | (° C.) (2) | 250° C. (%) |
| 1,3-butane diol | 171 | ND | ND |
| Nicotine | 190 | ND | 1.08 |
| 1,3-butane diol + | 175 | ND | ND |
| nicotine 10 mg/ml | | | |
| 20% GV + 80% PG | 162-209 | 301 | 0.65 |
| 40% GV + 60% PG | 162-233 | 303 | 1.64 |
| 50% GV + 50% PG | 166-239 | 304 | 0.85 |
| 60% GV + 40% PG | 162-243 | 303 | 1.19 |

(1) endothermic peak
(2) exothermic peak (reaction and/or decomposition)

Conclusions

The 1,3-butane diol is vaporized at a temperature of 171° C. without any thermal decomposition;

The nicotine is vaporized at a temperature of 190° C. while producing a residue after evaporation of 1.08 % associated with the presence in the nicotine of a non-vaporizable heavy compound;

In a mixture the 1,3-butane diol and the nicotine vaporize simultaneously at 175° C. (only 1 vaporization peak observed in TDA) without causing a decomposition and/or without reacting with one another.

Consequently, a formulation based on 1,3-butane diol and nicotine is perfectly adapted to ensure a constant delivering of the nicotine. In fact, a very different vaporization between the solvent and the nicotine would bring about in the course of time (that is, during the using of the electronic cigarette) a deterioration of the most volatile mixture in the compound and an enrichment of the heaviest compound. Under these conditions the concentration of the nicotine in the aerosol was not able to be constant in the course of the time. Finally, the 1,3-butane diol is a solvent of the thermally stable nicotine which vaporizes without interacting with the nicotine.

The totality of the formulations based on propylene glycol leads to the appearance in TDA of exothermic peaks characteristic for intermolecular reactions. There is therefore a hot chemical interaction (301-304° C.) between the constituents of the formulation;

The totality of the formulations based on propylene glycol leads to the presence of non-vaporizable residues at 350° C. corresponding to the formation of heavy secondary residues whose content is comprised between approximately 0.6 and 1.7%.

The thermal stability of the 1,3-butane diol as well as its absence of hot reaction with the nicotine shows its importance as a base for the formulation of liquid intended for personal vaporizers. In fact, this type of formulation does not generate when hot any volatile, toxic compounds such as formaldehyde, acetaldehyde, acrolein or any of the heavy secondary compounds, which is not the case for the current formulations based on propylene glycol and glycerol for which there is a real problem of technology and of innocuousness.

(1. Goniewicz & coll, Levels of Selected Carcinogens and Toxicants in Vapour from Electronic Cigarettes—TC Online First, published on 6 Mar. 2013 under

10.1136/tobaccocontrol-20-2012-050859). 2. Jensen, B. S.Wentai Luo, J. Pankow, R. M. Strongin,. D. H. Peyton. Hidden Formaldehyde in E-cigarettes Aerosols. New England Journal of Medicine, 372; 4, pp 392-3, 93, Jan. 35 22, 2015). 3. K. Bekki S. Uchiyama, K Ohta, Y. Inaba, H. Nakagome, N. Kunagita, Carbonyl Compounds, Generated from Electronic Cigarettes, Int. J. Environ. Res. Public Health 2014, 11, 11192-11200)

1. A method of preparing an electronic cigarette, the method comprising:
introducing an electronic cigarette e-liquid into a reservoir of an electronic cigarette, wherein
the electronic cigarette e-liquid includes a composition comprising a polyol, and
the polyol is a compound comprising 4 to 8 carbon atoms and 2 hydroxyl functions.

2. The method according to claim 1, wherein the polyol is a linear or cyclic compound.

3. The method of claim 1, wherein the polyol is a compound of vegetable or synthetic origin.

4. The method of claim 1, wherein the polyol is a compound selected from the group consisting of 1,2-butane diol, 1,3-butane diol, 1,4-butane diol, 1,2-pentane diol, 1,2-hexane diol, 1,2-heptane diol or 1,2-octane diol and mixtures thereof.

5. The method of claim 1, wherein the composition further comprises glycerol, the glycerol being present in an amount from 0 to 40% by weight of the composition.

6. The method of claim 1, wherein the composition further comprises propylene glycol.

7. The method of claim 6, wherein the propylene glycol is obtained from vegetable raw materials.

8. The method of claim 1, wherein the composition further comprises 1,3-propane diol.

9. The method of claim 1, wherein the composition further comprises one or more members selected from the group consisting of nicotine, a nicotine substitute and an aroma.

10. A liquid composition for an electronic cigarette, coniprising:
a polyol and at least one compound selected from the group consisting of nicotine, a nicotine substitute and an aroma, wherein the polyol is a compound comprising 4 to 8 carbon atoms and 2 hydroxyl functions.

11. The composition according to claim 10, wherein glycerol is not present in the composition.

12. The composition according to claim 10, further comprising propylene glycol.

13. The composition according to claim 10, wherein the composition does not comprise water.

14. An electronic cigarette comprising a composition according to claim 10.

15. A method of operating an electronic cigarette, the method comprising:
producing a vapor of an electronic cigarette liquid that is inhaled by a human user, wherein the electronic cigarette, liquid includes a long-chain polyol in an effective amount to
improve the tingling of the throat felt by a user and/or
improve the ease of inhaling the vapor.

16. The method of claim 15, wherein
the long-chain polyol is a polyol comprising 4 to 8 carbon atoms and 2 hydroxy functions, and
the electronic cigarette liquid comprises nicotine and an effective amount of the polyol comprising 4 to 8 carbon atoms and 2 hydroxyl functions to improve the bioavailability of the nicotine.

17. The method of claim 15, wherein the effective amount of the long-chain polyol is also effective to reinforce the aromatic potency.

18. The method of claim 15, wherein the effective amount of the long-chain polyol is also effective to limit or eliminate the formation of coproducts of thermolysis.

19. The method of claim 15, wherein the effective amount of the long chain polyol is also effective to augment the vapor density.
